(19) United States
(12) Patent Application Publication
(10) Pub. No.: US 2021/0338475 A1
(43) Pub. Date: Nov. 4, 2021

(54) Scalp Cooling Apparatus, System, and Method

(71) Applicant: Dignitana AB, Lund (SE)

(72) Inventors: William Cronin, Dallas, TX (US); Jim McKinney, Dallas, TX (US)

(21) Appl. No.: 17/246,256

(22) Filed: Apr. 30, 2021

Related U.S. Application Data
(60) Provisional application No. 63/018,113, filed on Apr. 30, 2020.

(51) Int. Cl. A61F 7/00

(52) U.S. Cl. CPC A61F 7/0085; A61F 7/007; A61F 2007/0078; A61F 2007/0056; A61F 2007/0096; A61F 2007/0008

(57) ABSTRACT

The present disclosure is directed to an apparatus, system, and method for the cooling and transferring of a fluid to a patient. The apparatus can be contained in an enclosure having at least two sections, that house an energy storage device, a power connection that may be coupled to an energy storage device; and a heat transfer assembly that cools the fluid. The enclosure may have a fluid receiving point, and a fluid transfer point with a sensor for monitoring the temperature of a fluid before transfer to a cooling wrap through a fluid transfer hose. A fluid transfer hose may couple the fluid transfer point at one end to a cooling wrap or cap at a second end. The fluid can be returned or received from the cooling wrap at the fluid transfer point, and the temperature of the fluid monitored when received at the fluid transfer point.

### CROSS-REFERENCE TO RELATED APPLICATIONS

This application claims benefit of U.S. Provisional Application No. 63/018,113 filed Apr. 30, 2020, which is incorporated herein by reference in its entirety. U.S. patent application Ser. No. 16/679,722 filed Nov. 11, 2019 and to be issued as U.S. Pat. No. 10,993,830, is incorporated herein by reference in its entirety.

### BACKGROUND

### Technical Field

The present disclosure relates to a device for scalp cooling. More particularly, and not by way of limitation, the present disclosure is directed to an apparatus, system and method for cooling a scalp during medical treatments.

### Description of Related Art

Cancer is one of the most vicious and life destroying diseases of our time. While there are treatments to reduce the effects and delay the end of life, these treatments are often times just as bad or worse than the actual disease.

### BRIEF SUMMARY

The present disclosure is directed to an apparatus for the cooling and transferring of a fluid. The apparatus can be contained in an enclosure having at least two sections. The enclosure may house at least one energy storage device, at least one power connection that may be coupled to the at least one energy storage device; and at least one heat transfer assembly. On one side of the enclosures there may be at least one fluid receiving point, and at least one fluid transfer point. The transfer point may have at least one sensor.

Thus, in one aspect, the present disclosure is directed to a system for the cooling and transferring of a cooling fluid. The system includes an enclosure sized and configured to house at least one energy storage device; at least one power connection, at least one heat transfer assembly, at least one fluid receiving point along a side, top or bottom of the enclosure, and at least one fluid transfer point along a side, top or bottom of the enclosure. The fluid transfer point may have at least one sensor. A fluid transfer hose may couple to the fluid transfer point at one end and a cooling wrap or cap at a second end.

In another aspect, the present disclosure is directed to a method of cooling and transferring a fluid. A system or device may have a fluid receiving point for filling the system or device with the fluid. The fluid is cooled with at least one heat transfer assembly. The temperature of the fluid is monitored with at least one sensor prior to a transferring the fluid to a cooling wrap through the fluid transfer hose. The fluid can be returned or received from the cooling wrap at the fluid transfer point, and the temperature of the fluid monitored when received at the fluid transfer point.

### BRIEF DESCRIPTION OF THE DRAWINGS

The novel features believed characteristic of the disclosure are set forth in the appended claims. The disclosure itself, however, as well as a preferred mode of use, further objectives and advantages thereof, will be best understood by reference to the following detailed description of illustrative embodiments when read in conjunction with the accompanying drawings, wherein:

FIG. 1 is an illustration of a scalp cooling system.

FIG. 2A is a front perspective view illustration of a cooling apparatus.

FIG. 2B is a rear perspective view illustration of a cooling apparatus.

FIG. 2C is an exploded front perspective view illustration of a cooling apparatus.

FIG. 3 is a side view illustration of a cooling apparatus.

FIG. 4 is a back view illustration of a cooling apparatus.

FIG. 5 is an illustration of a therapy hose and installation hose.

FIG. 6 is an illustration of a card reader and a card.

FIG. 7 is a schematic view drawing of a cooling wrap or cap.

FIG. 8 is an instructive illustration of one possible embodiment of a cooling wrap or cap being placed on a patient's head and/or scalp.

FIG. 9 is an illustration of the thermal cap being engaged and/or positioned for use.

FIG. 10 is a schematic illustration of a cooling apparatus.

FIG. 11 is a flowchart representation of graphical user interface screen of the cooling system.

FIG. 12 is an illustration of the buttons and/or commands available with the cooling system or device.

FIG. 13A is an illustration of low priority alarm screen.

FIG. 13B is an illustration of a medium priority alarm screen.

### DETAILED DESCRIPTION

An embodiment of the disclosure will now be described. Cancer can cause significant hair loss, and the hair loss can be accelerated by treatments utilized with cancer patients. These treatments can be radiation, and chemotherapy. Chemotherapy in particular can cause a majority if not all of a patient's hair to fall out. However, during treatment if the scalp is cooled the amount of hair loss can be reduced. Accordingly, there is a need for an apparatus, system and method for cooling a patient's scalp during medical treatments.

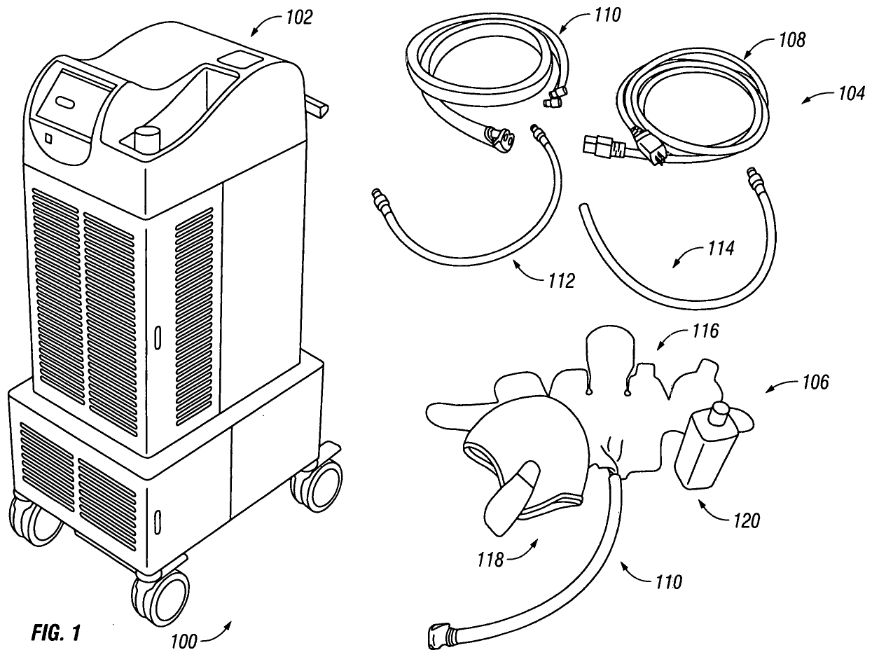

FIG. 1 is an illustration of a scalp cooling system 100. The scalp cooling system 100 can include a cooling apparatus 102, a set of connectivity devices 104, and the cooling caps and fluid 106. It would be understood that a set can include one or more of an item. The cooling apparatus 102 can cool a fluid provided to it, and transfer the fluid to a cap or wrap worn by a patient.

The cooling apparatus 102 can include an enclosure that allows for the housing and/or storage of cooling caps, fluid, connecting devices and/or portions of the cooling apparatus or system. The set of connectivity devices 104 can include a power cord 108, a therapy hose or a fluid transfer hose 110, an installation hose 112, and/or a drain hose 114. The power cord 108 allows for the coupling and/or connecting of the cooling apparatus 102 with a power source such as an Alternating Current (AC) source or Direct Current (DC) source. The therapy hose 110 allows for the transfer of a fluid or cooling fluid from the cooling apparatus 102 to a cap or wrap worn by a patient. In at least one embodiment, the therapy hose 110 may allow for both a transfer to, and a transfer from a cap or wrap worn by a patient. The cooling apparatus 102 may also have an installation hose 112. The installation hose 112 can allow for the testing and/or coupling of the cooling apparatus 102 to the cap or wrap worn by a patient. The drain hose 114 can allow for the fluid or cooling fluid to be drained from the cooling apparatus 102 and/or a reservoir (not illustrated) housed within the cooling apparatus 102.

The cooling caps and fluid 106, can include a cooling cap or wrap 116, a thermal cap or wrap 118, and/or the fluid or cooling fluid 120. The cooling cap or wrap 116 can have at least one channel that traverses the cooling cap or wrap 116 to allow for the fluid or cooling fluid 120 to travel through the cooling cap or wrap 116. In at least one embodiment, the cooling cap or wrap 116 can be coupled to the therapy hose 110. The thermal cap or wrap 118 can be placed over the cooling cap or wrap 116 to provide a tighter and/or comfortable fit to the patient's scalp. The fluid or cooling fluid 120, in at least one example, can be a glycol or alcohol based fluid.

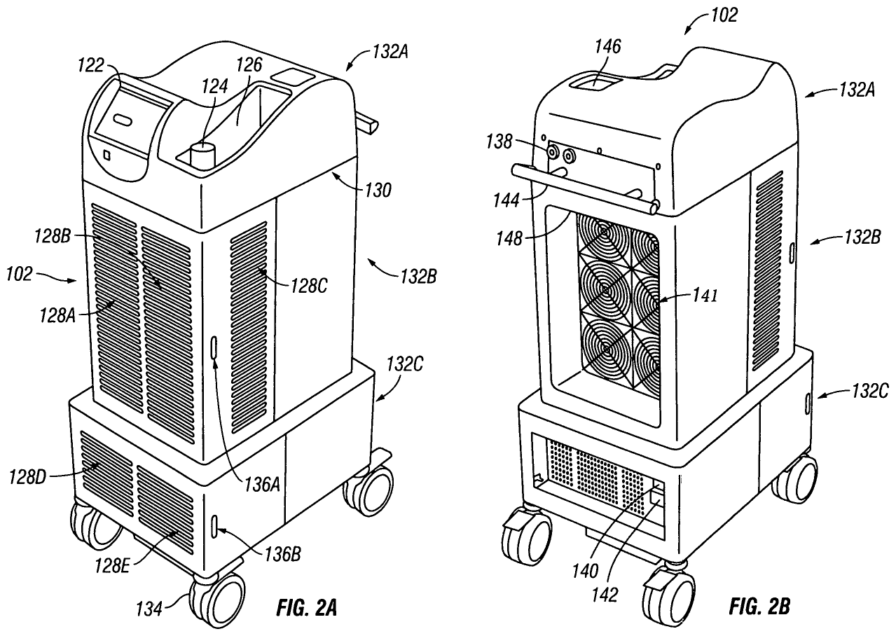

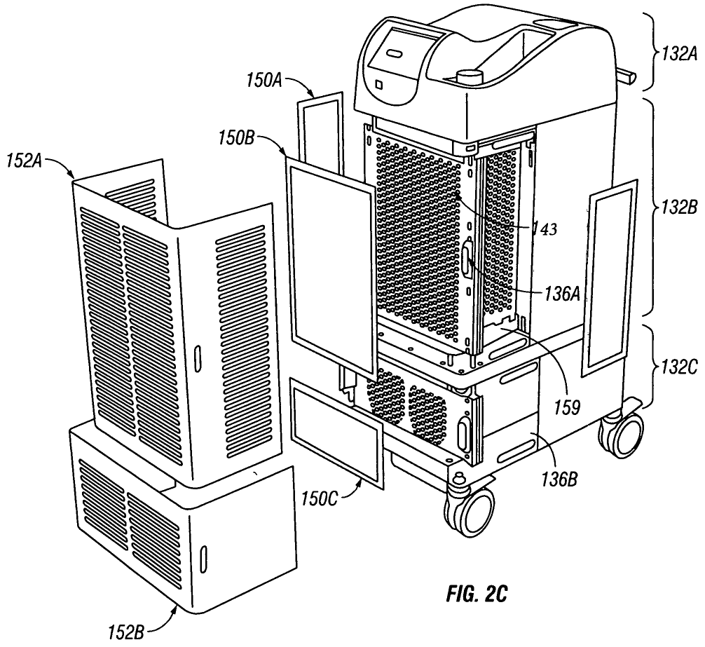

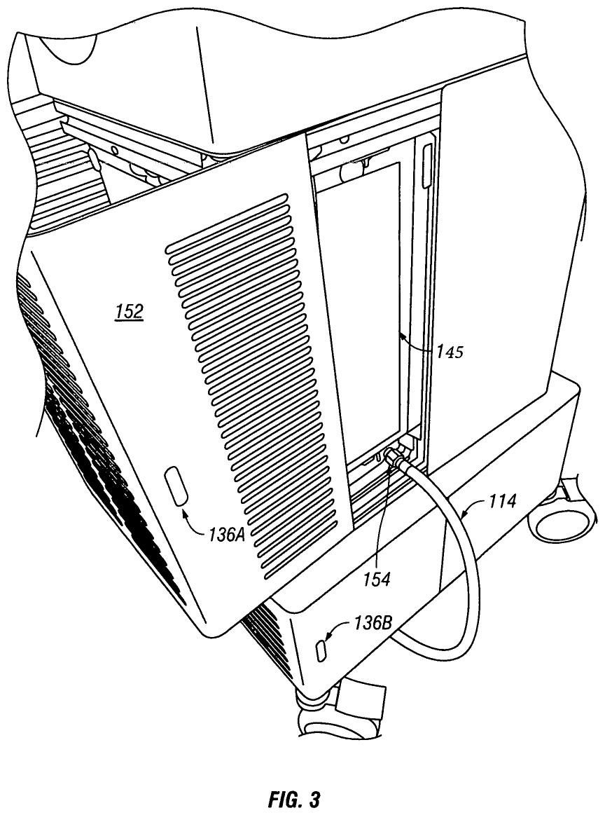

FIG. 2A is a front perspective view illustration of a cooling apparatus 102. The cooling apparatus 102 cools, prepares, and/or transfers fluid to other devices for use with patients. The cooling apparatus 102 can be operated via a user interface device 122, that can allow for the activation, deactivation, pump control, and/or cooling controls. A fluid reservoir fill point or fluid receiver point 124 can be affixed and/or coupled to the cooling apparatus 102. In at least one embodiment, the fluid reservoir fill point 124 can have an overfill vessel 126. The overfill vessel 126 can surround and/or encompass the fluid reservoir fill point or fluid receiver point 124 to allow for any spills and/or overflow can be collected and/or utilized by the cooling apparatus. The cooling apparatus 102 can also have vent(s) 128A, 128B, 128C, 128D, and/or 128E (collectively vents 128). In at least one embodiment, the vents 128 may traverse the side 130 of the cooling apparatus 102 and/or enclosure.

The cooling apparatus 102 can have an upper section 132A, a mid-section 132B, and/or a lower section 132C. In some examples, the cooling apparatus 102 may have no visible sections. The upper section 132A, in at least one embodiment, can house the user interface 122, the fluid reservoir fill point 124, and/or the overfill vessel 126. The mid-section 132B can house and/or include a set of heat transfer units, fluid reservoirs or tanks, pump(s), control system(s), and/or other structural or fluid devices. The lower section 132C can house power system(s), energy storage device(s), a set of heat transfer units, fluid reservoirs or tanks, pump(s), control system(s), and/or other structural or fluid devices. The lower most section, may also be coupled to a set of casters or roller(s) 134. The set of casters or roller(s) 134 could allow for the movement of the cooling apparatus 102. The side(s) 130 of the cooling apparatus 102 can be removed when release button(s) 136A and/or 136B are pressed. In at least one example, the mid-section 132B can have removable side(s) 130 and/or the lower section can have removable side(s) 130.

FIG. 2B is a rear perspective view illustration of a cooling apparatus 102. The cooling apparatus 102 utilizes heat transfer assemblies (not illustrated) to cool fluids to a desired temperature. The cooling apparatus 102, in at least one example, can have three sections, a top or upper section 132A, a mid-section 132B, and a bottom or lower section 132C. The top section 132A can house various control systems, and/or user interfaces such as a card reader 146. In at least one embodiment, the top section 132A may also provide support for a handle 144. In some examples, the handle 144 may be utilized for pushing or pulling the cooling apparatus 102 from different rooms to allow for additional patient use. The mid-section 132B can house various control systems and operational components, including but not limited to fluid storage. Additionally, the mid-section 132B can also house the heat transfer assemblies (not illustrated), which may require fans 141 to assist in cooling a fluid. The mid-section 132B may also have an aperture 148 that allows for the fans 141 to be recessed and housed away from any clothing and/or material. The heat transfer assemblies (not illustrated) can reduce the fluid temperature in incremental stages and be measured in Celsius or Fahrenheit. The lower section 132C, in at least one example, can house the power and/or power control system include power switch and/or breaker 140 and/or a power cord connection point 142. In some examples, the lower section 132C may also include fan units as well.

FIG. 2C is an exploded front perspective view illustration of a cooling apparatus 102. As previously discussed, the cooling apparatus can have three sections, an upper section 132A, a mid-section 132B, and a lower section 132C. Each of these sections may incorporate filtering technology, such as but not limited to, filter(s) 150A, 150B, and/or 150C (collectively filters 150). These filters 150 may, in at least one example, be held in place by screen(s) 143. The screens 143 can act as a barrier to prevent the filters 150 from getting caught and/or effecting the operations of the cooling apparatus 102. The filters 150, in at least one embodiment, would meet and/or exceed High-Efficiency Particulate Air (HEPA) filter standards. In at least one example, the filters 150 may also operate in combination with a medical grade air purification system 159. The filters 150 may also be held in place by outer cover(s) 152A and/or 152B (collectively outer covers 152). In at least one embodiment, the outer covers 152 are removable when a release button 136A and/or 136B is pressed and/or depressed. The release button(s) 136 may be found on the screens 143 or on another location of the cooling apparatus 102.

FIG. 3 is a side view illustration of a cooling apparatus 102. The cooling apparatus 102, in at least one embodiment, is utilized for the cooling of a fluid for use with medical devices. The fluid can be stored in a fluid or storage reservoir 145 that is housed within the cooling apparatus 102. In at least one embodiment, the fluid or storage reservoir 145 is housed in the mid-section (not illustrated) of the cooling apparatus 102.

The fluid or storage reservoir 145, in at least one example, can be accessed by a user, or medical professional by removing the outer cover 152. The outer cover 152 may be removed during and/or after a release button 136A and/or 136B are pressed or depressed. The fluid or storage reservoir 145 may also have a drain or drainage point 154. The drain or drainage point 154 can be coupled to a drain hose 114. The drain hose 114 can be utilized to drain a fluid or cooling fluid into a container for transport, and/or disposal.

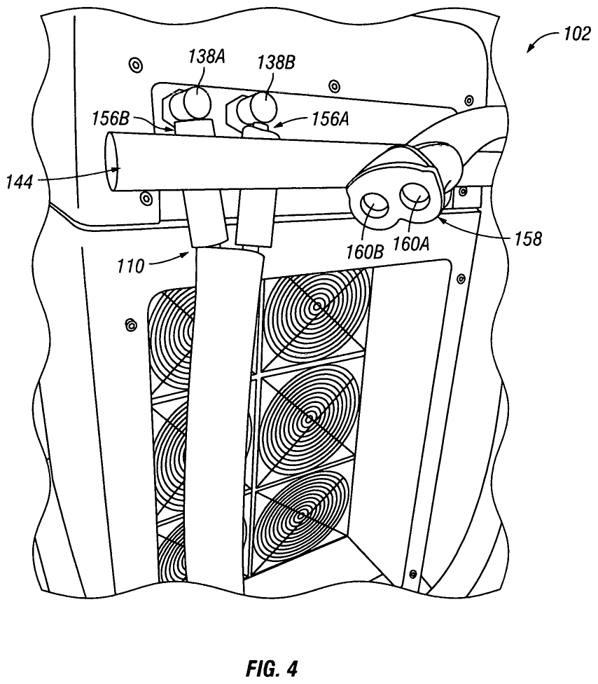

FIG. 4 is a back view illustration of a cooling apparatus 102. The cooling apparatus 102, in at least one embodiment, is utilized for the cooling of a fluid for use with medical devices. The fluid (not illustrated) can be transferred and/or transported from the cooling apparatus 102 by the fluid transfer point(s) 156A and/or 156B (collectively fluid transfer point 156). In at least one example, the fluid transfer point 156A can be utilized for transfer to an external device, while fluid transfer point 156B can be utilized for the return of a fluid from an external device. The fluid transfer point(s) 156 may be mechanically and/or fluidly coupled to a therapy hose 110. The therapy hose 110 can have at a first end a set of therapy hose connection(s) 138A and/or 138B (collectively therapy hose connections 138). In at least one embodiment, the therapy hose connections 138 can allow for a positive or transfer from (connection 138A) the cooling apparatus 102, and a negative or return to (connection 138B) the cooling apparatus 102. It would be understood that both connections can allow for positive or transfers from the cooling apparatus 102, as well as negative or returns to the cooling apparatus 102, additional connections and/or a combination thereof. The therapy hose 110 may also have at a second end, a therapy device connection 158. The therapy device connection 158 may have, in at least one example, a set of therapy device connection points 160A and/or 160B that allow for mechanical and/or fluid connection to a therapy device. A mechanical and/or mechanically connected devices are connected through a mean of connection such as but not limited to, a screw or turn style connection, a turn style locking connection, and/or a friction based connection. The set of therapy device connection points 160A and/or 160B can allow for positive or transfers to a therapy device (not illustrated), as well as negative or returns from a therapy device, additional connections and/or a combination thereof. In at least one embodiment, the cooling apparatus 102 can also have handle 144.

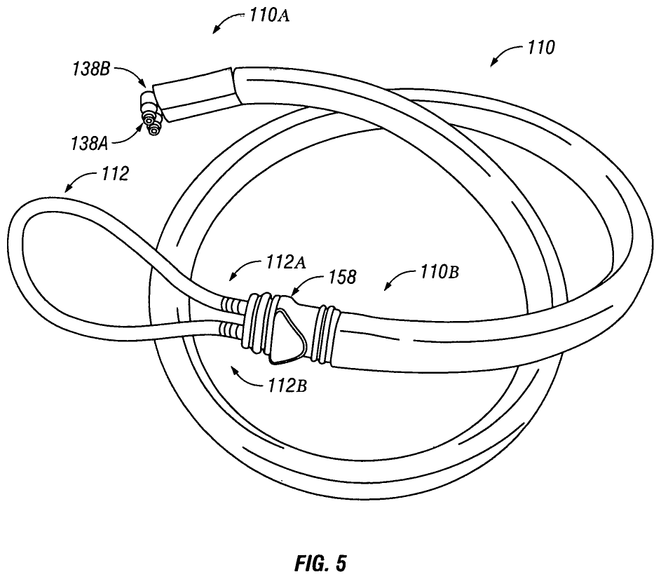

FIG. 5 is an illustration of a therapy hose 110 and installation hose 112. A therapy hose 110 can have a first end 110A and a second end 110B. At the first end 110A the therapy hose 110 may have a set of therapy hose connection(s) 138A and/or 138B (collectively therapy hose connections 138). The therapy hose connections 138 can allow for a mechanical and/or fluid connection with a cooling apparatus (not illustrated). For example, the therapy hose connections 138 may have a screw like mechanism along the outer portion of the connections that allows the connections to be securely fastened to the cooling apparatus (not illustrated), and also allow for fluid to flow from the cooling apparatus into the therapy hose 110 through an internal portion of the connections similar to a pipe. At the second end 110B of the therapy hose 110, there can be a therapy device connection 158. The therapy device connection 158 may be coupled with a therapy device (not illustrated) or an installation hose 112. The installation hose 112 can have test connection point(s) 112A and/or 112B that can be coupled to the therapy device connection 158. The installation hose 112 can allow for the testing of the cooling apparatus 102 without utilizing a therapy device.

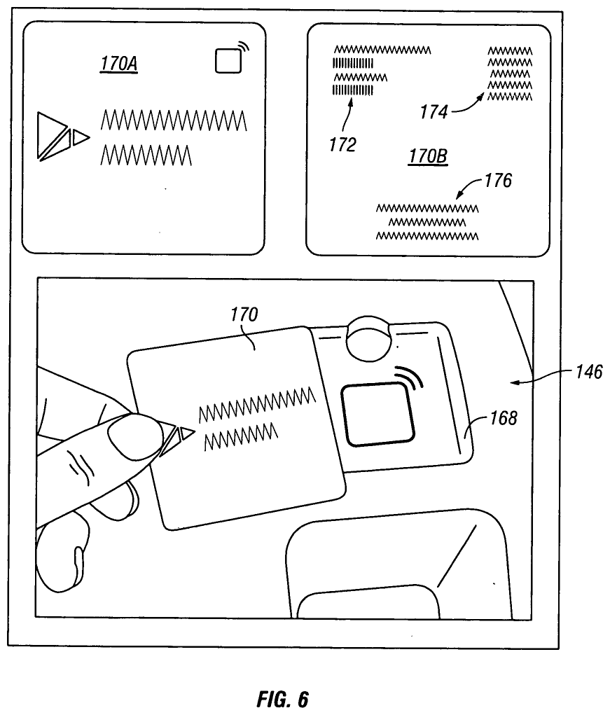

FIG. 6 is an illustration of a card reader 146 and a card 170. The card reader 146 may be coupled to a control system and/or control circuitry (not illustrated) to allow for access to various aspects of the cooling apparatus (not illustrated). The card reader 146 may include the ability to read, and/or scan a card 170 through a near field communication (NFC), an optical scan, a magnetic scan, Bluetooth, zigbee, wifi, and/or other communication protocols. In at least one embodiment, the card reader 146 is an NFC reader with an indention 168 that allows the card 170 to be placed and/or rest while a user utilizes the cooling apparatus, and/or accesses the control system of the cooling apparatus. The card 170 can have a front side 170A, and a back side 170B. It would be understood that the sides could be interchangeable. In some examples, one side of the card 170 may have a set of codes 172, such as but not limited to bar codes, QR codes, and/or other types of identifying code like structures. There can be text 174 that allows for warnings or other information, and/or text 176 such as but not limited to identifying information about a facility or a patient. As a note, if for a patient, the information would likely be limited to last name or first name only or other non-identifying information such as a patient id number.

For example, the card reader 146 may detect when a user or medical professional (not illustrated) has placed, swiped, and/or scanned the card 170. If the card 170 is one that is recognized by the system and/or is an approved card 170 then the user can access the cooling apparatus, and/or the control system. The control system may allow for the user or medical professional to access portions of the cooling apparatus, such as the fluid reservoir, and/or the heat transfer assemblies. In other examples, the control system when accessed would allow a user to set the duration, and/or temperature of the cooling cycle.

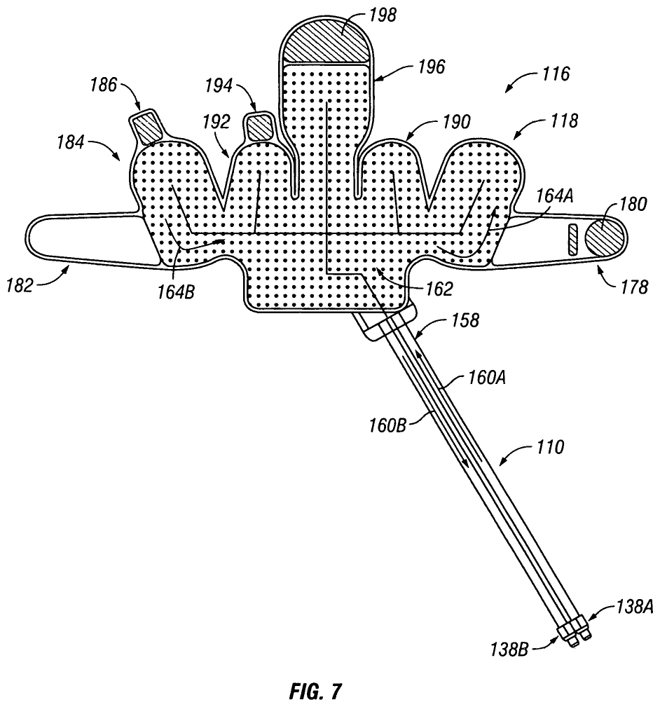

FIG. 7 is a schematic view drawing of a cooling wrap or cap 116. In some examples, the cooling wrap or cap 116 can be a therapy device. The cooling wrap or cap 116 can be coupled to a therapy hose 110. The therapy hose 110 can have a therapy device connection 158 at one end, and therapy hose connection(s) 138A and/or 138B (collectively therapy hose connections 138) at the other end. The therapy hose 110 allows for a fluid to be supplied 160A from, and/or returned 160B to a cooling apparatus (not illustrated).

As the fluid flows 164A and/or 164B through the channel 162 of the cooling wrap or cap 116, it can pass through multiple portions or petals of the cooling wrap or cap 116. For example, a cooling wrap or cap 116 can have a first strap portion 178 and a second strap portion 182. In at least one example, when placed on a patient's head or scalp (not illustrated) a fastener or fastening point 180 on the first strap portion 178 can be coupled with a corresponding fastener or fastening point (not illustrated) on the second strap portion 182. The fastener or fastening point 180 may be a hole and button, a hook and loop, a snap, a belt like interaction, and/or other means of fastening.

A first lower petal 184 may interact and/or engage with a second lower petal 188 through a fastener or fastening point 186. The first lower petal 184 and/or the second lower petal 188 may couple with one and/or both of the first strap portion 178 and the second strap portion 182. A first upper petal 192, and a second upper petal 190 may interact via a fastener or fastener point 194. In at least one example, the first upper petal 192 and/or the second upper petal 190 may interact or couple with the first lower petal 184 and/or the second lower petal 188. A closing tab 196 may interact with any of the petals or strap portions via a fastener or fastening point 198.

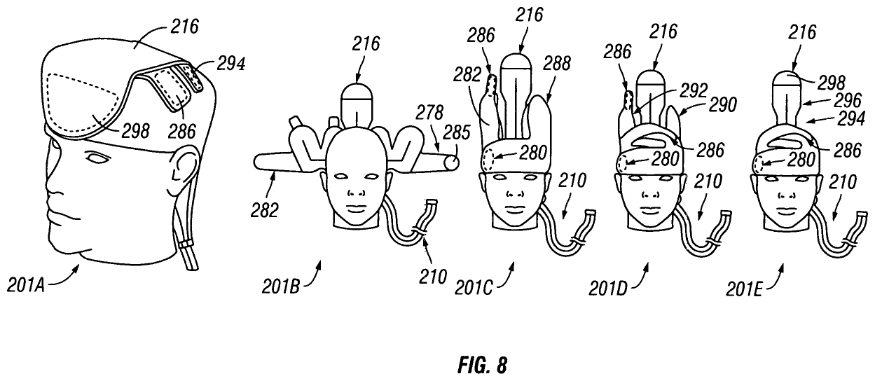

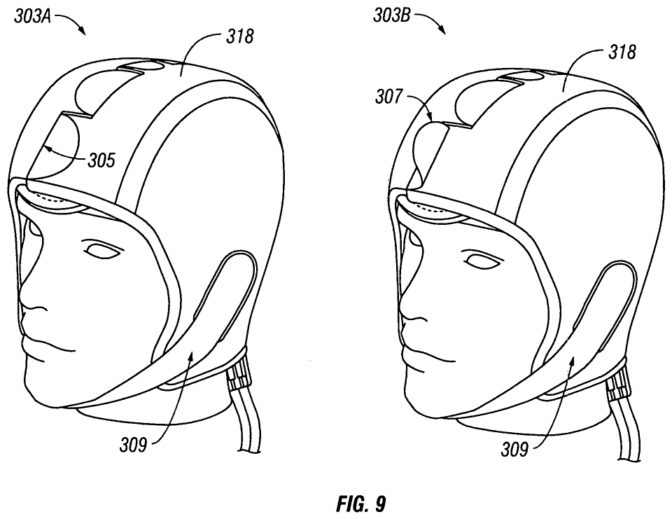

FIG. 8 is an instructive illustration of one possible embodiment of a cooling wrap or cap 216 being placed on a patient's head and/or scalp. For example, view 201A is an illustration of the cooling wrap or cap 216 being fully engaged with a patient's head or scalp. The fasteners 298, 286, and/or 294 are all engaged with a corresponding fastener or fastener point.

View 201B illustrates the cooling wrap or cap 216, just before the first strap portion 278 is engaged with the second strap portion 282 via the fastener 280. The strap portions 278 and 282 may be aligned with certain portions of a patient's head or scalp in order to provide steady and/or consistent contact across the entire surface to be cooled and/or treated via the therapy hose 210.

View 201C is an illustration of the cooling cap or wrap 216 partially engaged by the fastener 280. The first lower petal 284 and the second lower petal 288 can be prepared for engagement via the fastener 286. When the fastener 280 is engaged, the lower petals 284 and/or 288 can be positioned to cover the front portion of a patient's scalp or head.

View 201D is an illustration of the cooling cap or wrap 216 partially engaged by the fasteners 280 and 286. The first upper petal 292, and the second upper petal 290 can be positioned to cover the rear of a patient's head or scalp. View 201E is an illustration of the cooling cap or wrap 216 before the closing tap 296 is secured via the fastener 298. The upper petals are secured by fastener 294 and the lower petals are secured by fastener 286. Once the closing tab 296 is closed the cooling cap or wrap 216 can be utilized for therapy purposes.

FIG. 9 is an illustration of the thermal cap 318 being engaged and/or positioned for use. In view 303A the securing slot 305 can receive a securing flap 307 as seen in view 303B. The securing flap 307 and securing slot 305, can allow for the thermal cap 318 to be secured in a side to side manner (ear to ear). A chin strap 309 can also allow for the securing of the thermal cap 318 in an up and down manner.

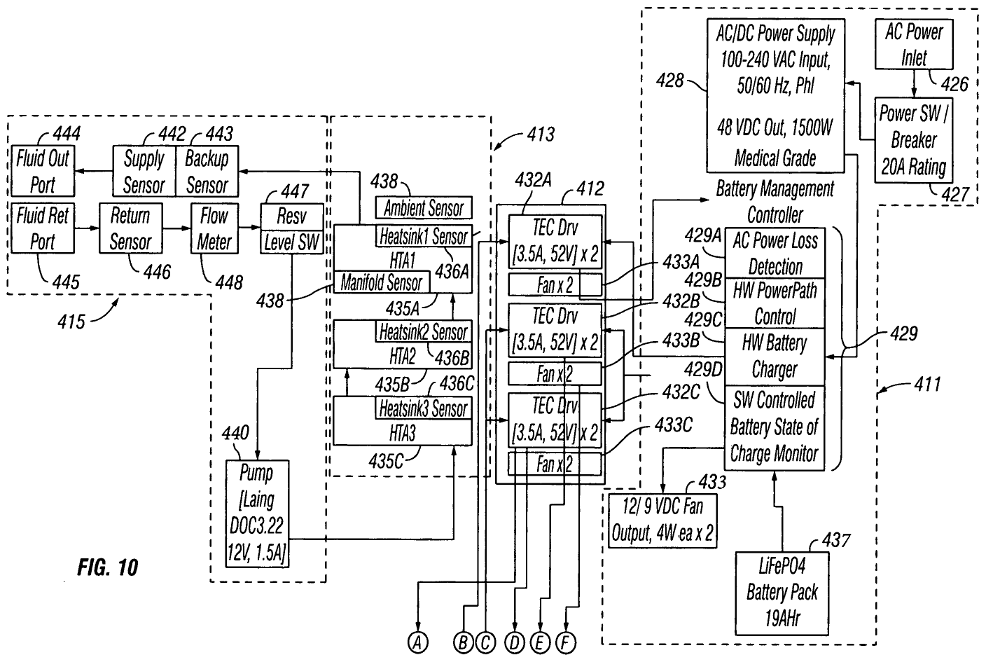

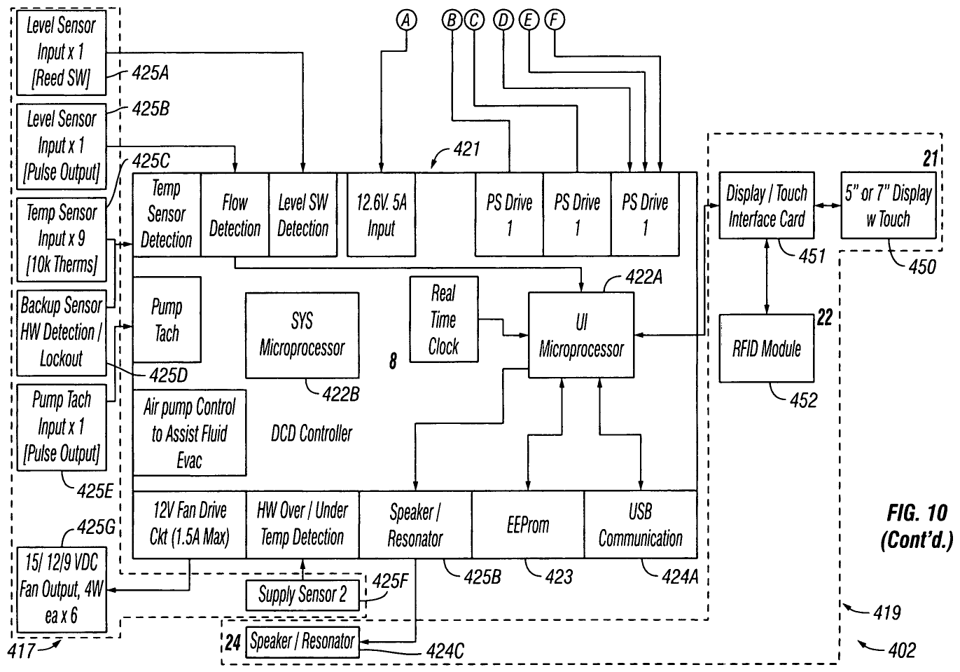

FIG. 10 is a schematic illustration of a cooling apparatus 402. The cooling apparatus 402 can have a power unit 411, a fan unit 412, a temperature modification unit 413, a fluid sensor and control unit 415, a sensor unit 417, and a user interface unit 419, which can be controlled and/or operated by a computing device 421. A computing device 421 can include a set of processor(s), a set of memory devices, a set of storage devices, at least one user interface device, and other devices capable of being utilized by or supporting the operations of the computing device 421. In at least one embodiment, the computing device 421 is a micro-controller or microprocessor 422A and/or 422B that is coupled to a set of memory 423, and user interface devices 424A, 424B, and/or 424C.

The computing device 421 may also be coupled to a sensor unit 417. In at least one example, the sensor unit 417 may include multiple sensor(s), such as but not limited to, a level sensor 425A, a flow sensor 425B, a temperature sensor 425C, a set of backup sensor(s) 425D, a pump tachometer sensor 425E, a supply sensor 425F, and/or a fan drive sensor 425G. In some examples, the fan drive sensor is incorporated with the fan drive.

The computing device 421 may also be coupled to a power unit 411. The power unit 411, in at least one embodiment, can include, a power supply input 426, a breaker 427, a power supply 428, a power system controller 429, a set of batteries 437, and at least one fan 431. In at least one example, the power system controller 429 can include a power loss detection unit 429A, a power path control device 429B, a battery charger 429C, and/or a state of charge monitoring device 429D.

A fan unit 412 may be coupled to the power unit and/or the computing device 421. The fan unit 412 may include fan drivers 432A, 432B, and/or 432C, and/or fan(s) 433A, 433B, and/or 433C. The fan unit 412 can assist in removing heat and/or cool air from the area surrounding the temperature modification unit 413. In at least one embodiment, the temperature modification unit 413 may include at least one heat transfer assembly 435A, 435B, and/or 435C (collectively 435). The heat transfer assemblies 435 can include, in at least one example, a heat sink sensor 436A, 436B, and/or 436C. In some examples, the heat transfer assemblies 435 may also include a manifold sensor 437 and/or an ambient sensor 438. The manifold sensor 437 can be used to detect the temperature of a fluid moving through pipes and/or tubing coupled to the heat transfer assemblies 435. The heatsink sensor(s) 436 may be utilized to know the temperature difference between the fluid (not illustrated), the ambient temperature, and/or the heatsink.

In at least one example, the heat transfer assemblies 435 are thermo-electric panel manufactured by ThermoTek, Inc. The thermo-electric panels allow for thermo-electric coupling effects to reduce the temperature of a fluid passing through piping or conduit housed within the thermo-electric panel. Thermo-electric coupling effects allow for the transfer of heat based on voltage differences between panels. The thermo-electric panels utilized in the heat transfer assemblies, include piping or conduits that pass through the heat transfer assemblies 435 and the thermo-electric panels which cause the fluid to be cooled based on the manifold sensor 437 and/or an ambient sensor 438. For example, the heat transfer assemblies may require additional power to cool a fluid if operated in an environment having an ambient temperature of eighty degrees Fahrenheit. Further to this example, the heat transfer assemblies 435, the manifold sensor 437 can be utilized to adjust the power applied to the thermo-electric panels to further cool them. In at least one example, the heat transfer assemblies 435 employing thermo-electric panels can allow for reduced condensation and/or excess heat generated from the cooling operation over a condenser based system.

The fluid sensor and control unit 415 can allow for the monitoring and controlling of fluid movement through, from and/or to the cooling apparatus 402. The fluid sensor and control unit 415, in at least one example, may include a pump 440 for moving the fluid, a reservoir 441 for storing the fluid, a supply sensor 442 for detecting when fluid is available for use, a backup sensor 443, a fluid output port 444 to provide a connection point and/or exit point for the fluid to a therapy device, a fluid return port 445, a return sensor 446 to detect when fluid is returning, and/or a flow meter 448 to determine how much fluid has exited and/or returned to the reservoir 441.

In at least one embodiment, the utilization of heat transfer assemblies 435 are utilized to keep the temperature with one degree Celsius or Fahrenheit of the desired temperature. Additionally, rather than place the supply sensor 442 and/or return sensor 446 in the cooling cap or wrap (not illustrated) as in previous designs, the sensors 442 and/or 446 are placed within the cooling apparatus 402. This is advantageous for multiple reasons, first because the sensors 442 and/or 446 are critical to maintaining and/or controlling the temperature as felt and/or received by the patient the accuracy and integrity of the sensors 442 and/or 446 should remain at a consistent level. Second, previous designs allowed for sensors 442 and/or 446 that were placed within the cooling cap or wrap to become damaged during preparation and/or conclusion of a treatment session as the cooling cap or wrap was handled by the patient and/or medical personal. As a result, the consistency of the cooling and/or therapy treatment were degraded. Thus, in order to increase consistency, the placement of the sensor(s) 442 and/or 446 within the cooling apparatus 402 provides increase in the effectiveness and/or efficiency of the sensor(s) 442 and/or 446. In at least one example, the sensor(s) 442 and/or 446 may also improve over previous designs because of the ability to have a less compact design, increasing reliability and accuracy. Additionally, the sensors 442 and 446 can also be placed in line with the fluid flow allowing for additional accuracy of fluid temperature readings. Previous designs measured the temperature of a user's scalp rather than the fluid temperature allowing for multiple changes to be read. For example, a user drinking a warm liquid may cause the scalp temperature to temporarily rise, and if readings are based on scalp temperature the system may decrease the fluid temperature accordingly to a point of cause more harm than good. Whereas, if the sensors 442 and 446 are utilized in an in-line configuration then the fluid temperature can be determined and adjusted based on the heat transferred from the scalp to the fluid. The sensors 442 and/or 446 may also incorporate flexible sensors cores to allow for flexibility when hoses are coupled to the cooling apparatus or system.

The computing device 421 may be coupled to a user interface unit 419. In at least one example, the user interface unit 419 may include a display 450, a display and/or touch interface control unit 451, a card reader 452, and/or audio devices such as a microphone or speaker 424C. The display 450, if it is a touchscreen, may include and/or couple to a display and/or touch interface control unit 451 to allow for the detection and corresponding translation to a signal capable of begin utilized by the computing device 421. A card reader 452 may also be coupled to the computing device 421. The card reader 452 may include a Radio Frequency Identification (RFID), NFC, Bluetooth, wifi, zigbee, magnetic, and/or optical reading and/or scanning technology. The card reader 452 can allow a user to access the control system and/or settings for operational and/or maintenance purposes.

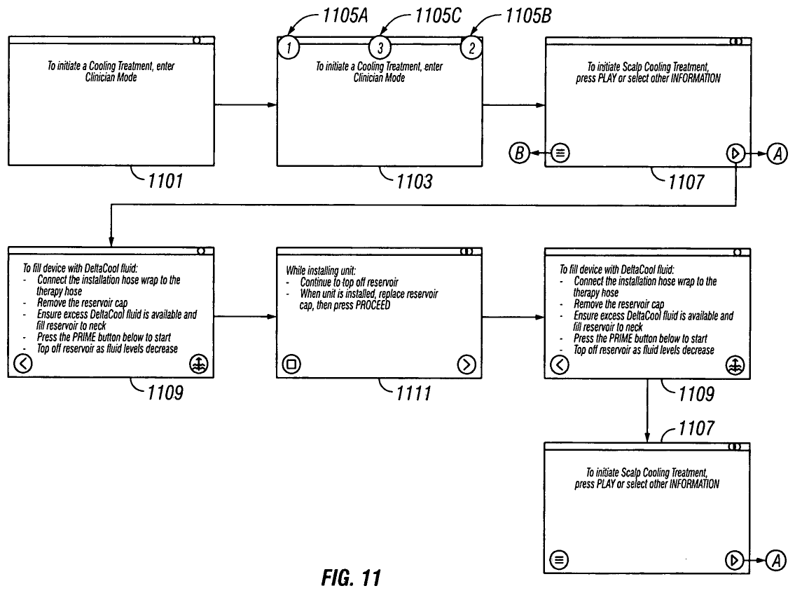

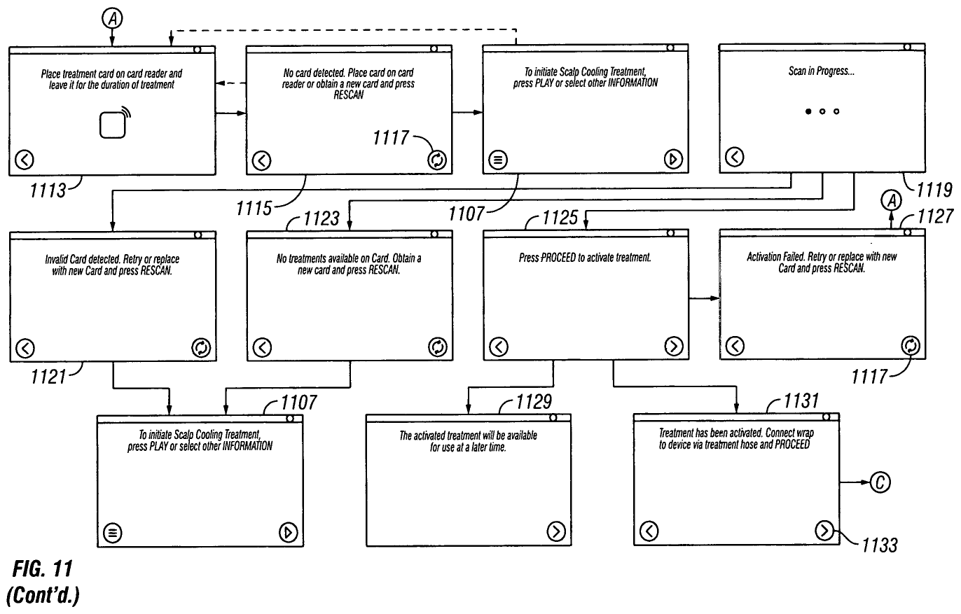

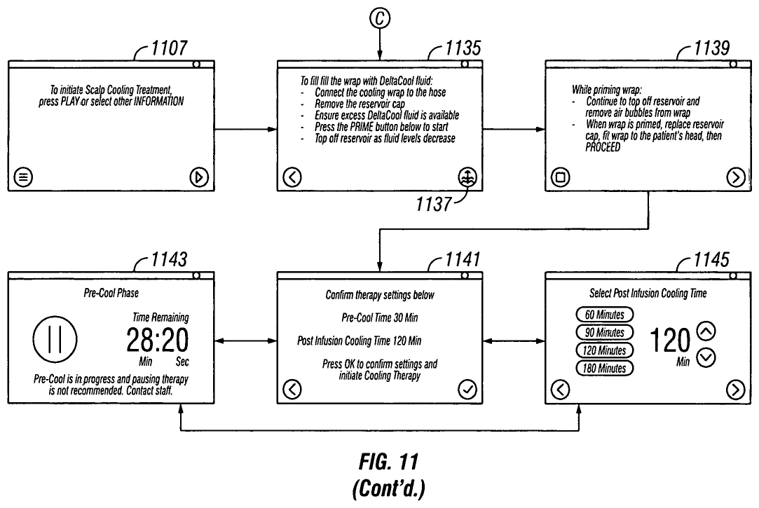

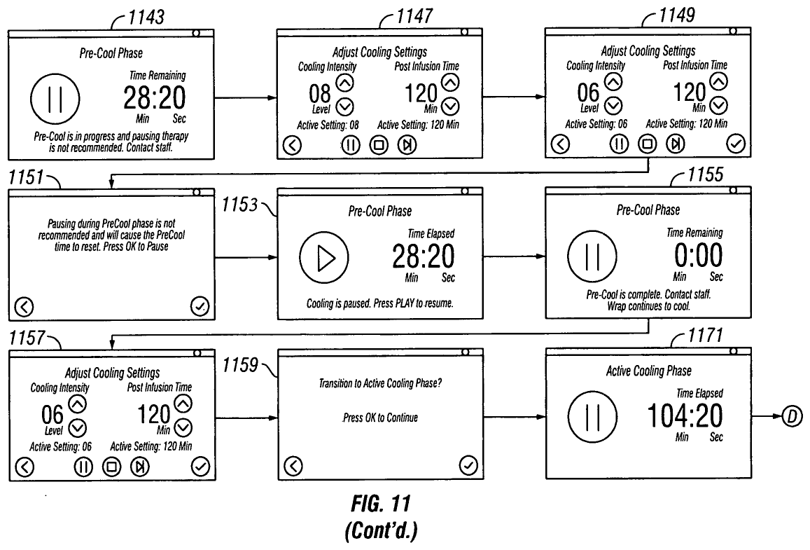

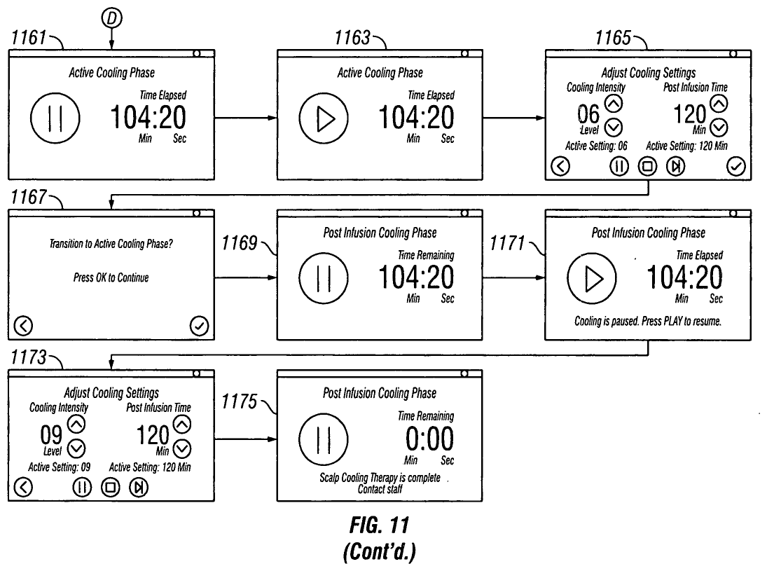

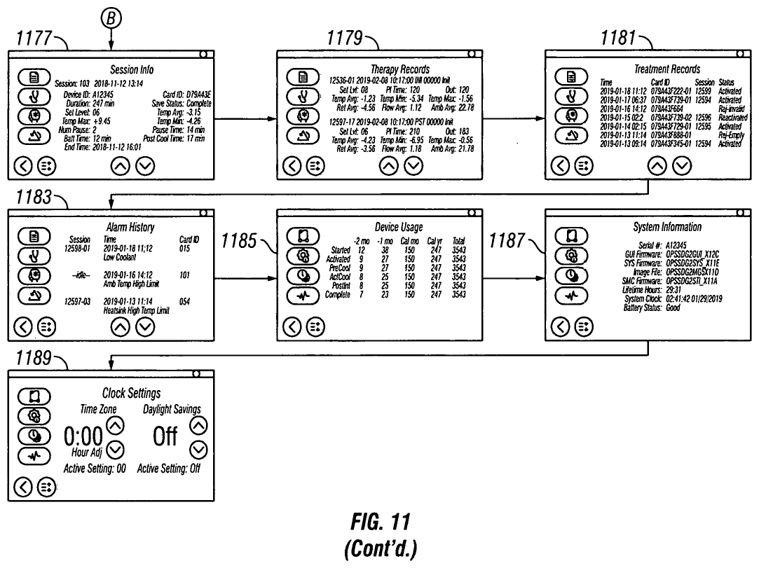

FIG. 11 is a flowchart representation of graphical user interface screen of the cooling system. The graphical user interface, in at least one example, operates from computer readable mediums, and proprietary software for controlling and/or operating the cooling system or apparatus. Upon powering the cooling system, a patient and/or physician or other medical professional could be presented with the treatment initiation screen 1101. In at least one embodiment, the treatment initiation screen 1101 can include a prompt to enter a clinician mode to initiate a treatment. In at least one example, the treatment initiation screen 1101 may also have a patient information or patient portal access point or button (not illustrated).

A physician or other medical professional can access the clinician mode through an access code screen 1103. In at least one embodiment, the access code screen 1103 may be similar to the treatment initiation screen 1103. In at least one example, the access code screen 1103 may also have additional functionality or buttons from the treatment initiation screen 1103. For example, the access code screen 1103 can also include one or more buttons that would have a specified entry code such as, but not limited to a first button 1105A, a second button 1105B, and a third button 1105C (collectively buttons 1105). In this example, the first button 1105A could be on the far left of the screen, the second button 1105B may be on the far right of the screen, and the third button 1105C can be in the middle of the screen. These buttons 1105, could be at the top, bottom, left, right, middle, or any combination thereof. If the entry of the specified access code is correct, the physician or other medical professional would be directed and/or provided a scalp cooling treatment screen 1107.

The scalp cooling treatment screen 1107 can allow for a physician or other medical professional to begin a treatment or system information. In at least one embodiment, the system information would also include system configurations, treatment configurations and/or alert configurations. To initiate a scalp cooling treatment, a physician or other medical professional may press a play button or other button (labeled as A) that would allow the system to initiate or begin a scalp cooling procedure. The system information would be accessed through an indicator or button (labeled as B). There are several different screens the scalp cooling treatment screen 1107 can lead to. As an example, if the scalp cooling system does not have any cooling fluid and/or has not been primed then a fill device screen 1109 may be presented.

The fill device screen 1109 can provide instructions on filing and/or priming the scalp cooling system and/or device with a cooling fluid. In at least one embodiment, the cooling fluid is a DeltaCool fluid that is comprised of an alcohol and water based fluid. In at least example, the fluid may utilize a glucose based fluid. Some of the instructions provided by the fill device screen 1109 could include: connecting the installation hose wrap to the therapy hose, removing the reservoir cap, filling the reservoir after ensuring there is available cooling fluid, priming the cooling system or device, and topping off the reservoir after priming. Additional instructions may be included on an installation screen 1111. The installation screen 1111 may be presented the first time there is a filling and/or if no scalp cooling cap or wrap is detected. The installation screen 1111 may then allow for a return to the fill device screen 1109. After the system and/or device has been filled and/or installed according to sensors and/or user input or feedback the system may return to the scalp cooling treatment screen 1107. If the play button (A) is selected the system or device may send the user and/or physician or other medical professional/provider to a treatment card screen 1113.

The treatment card screen 1113 may instruct a user how to utilize a patient treatment card or DeltaCard. The patient treatment card allows for treatment specifications and/or authorizations to be provided to the system or device. For example, the DeltaCard may allow for five treatment sessions, and/or provide specifications or settings for each of those sessions. In other examples, the DeltaCard allows for security by providing each patient with an anonymous method of unlocking a treatment and/or treatment plan without providing any patient details. In at least one example, the patient treatment card would also be linked to a physician and/or medical professional limiting who could interface with a user and/or patient and modify treatment plans accordingly.

In at least one embodiment, the treatment card screen 1113 prompts a user, patient, physician, and/or medical professional to place a patient treatment card on a reader and/or scanner of the system or device. If the reader or scanner fails to read or scan the patient treatment card then a no card detected screen 1115 may be provided. The no card detected screen 1115 may allow for a user to return to the treatment card screen 1113 or through a rescan button 1117 may allow a user, patient, physician, or medical professional to attempt a rescan of the patient treatment card. If the read, scan, reread, or rescan is successful, the system or device may return the user, patient, physician, or medical professional to the scalp cooling treatment screen 1107. If both a patient and physician or medical professional must authorize use of the cooling system or device, the scalp cooling treatment screen 1107 may return to the treatment card screen 1113 for additional reading or scanning.

In at least one embodiment, the system or device may detect that a patient treatment card during the treatment card screen 1113, but a not perform a reading or scanning of the patient treatment card until a scan in progress screen 1119 is provided. In at least one embodiment, the scan in progress screen is provided after a user, physician or medical professional initiates the scalp calling treatment from the scalp cooling treatment screen 1107. In at least one example, the scan in progress screen 1119 may be provided after the treatment card screen 1113. After completing the reading or scanning of the patient treatment card, there are many different screens that maybe provided based on conditions and/or rules of the system or device. In at least one example, theses conditions and/or rules may be based on patient information or that desired by a clinic or treatment facility.

Some of the screens that may be provided after the scan in progress screen 1119 could include an invalid card screen 1121, a no treatments available screen 1123, and/or proceed to active treatment screen 1125. These screens 1121, 1123, and/or 1125 may each have a rescan button 1117 that allows for the patient treatment card to be rescanned and/or reread because of an issue that may have occurred because of a physical issue or an issue related to software and/or hardware of the system or device. For example, the software may read the patient treatment card as invalid (invalid card screen 1121) because of a transposition of numbers or letters, when scanning the patient treatment card an error may occur where treatments are not available (no treatments available screen 1123), or everything may be perfect and ready for a treatment to begin (active treatment screen 1125). Both the invalid card screen 1121 and the no treatments available screen 1123 may proceed and/or return to the scalp cooling treatment screen if a rescan and/or reread is not successful. If successful, then both screens 1121 and 1123 may lead to the active treatment screen 1125.

From the active treatment screen 1125 a user, patient, physician, or medical professional may be directed and/or provided one of three different screens regarding the initiation of a treatment or treatment session. For example, if the activation of the treatment or patient treatment card fails, the user, patient, physician, or medical professional may have an activation failed screen 1127 presented to them. If the activation of the patient treatment card fails, a rescan button 1117 may be pressed to rescan or reread the patient treatment card. If the activation cannot be completed then the user, patient, physician, or medical professional may be returned to the treatment card screen 1113. If the activation is successful the user, patient, physician, or medical professional can be presented with a delayed treatment session screen 1129, and a proceed to treatment session screen 1131. Many of these screens may also have a directional button 1133A and/or 1133B that allows for forward and backward movement between the screens. In at least one embodiment, the directional button 1133A and/or 1133B would not allow a user to move forward to different screens unless allowed by the conditions and/or rules for the treatment, treatment session, user, patient, physician, medical professional, or facility. If for some reason the user or patient is not authorized and/or supposed to receive a treatment on the date or time in question, they will be provided or directed to the delayed treatment session screen 1129.

If it is the first time the user, patient, physician, or medical professional is utilizing the cooling system or device the system may need to be prepared. In particular, the user or patient's cooling cap or wrap may need to be filled and/or primed. In at least one embodiment, a fill wrap screen 1135 may be provided and/or directed to after the scalp cooling treatment screen 1107 or treatment session screen 1131. The fill wrap screen 1135 can provide instructions to a user, patient, physician, or medical professional on how to fill the cooling wrap or cap, along with connecting the hoses, and priming the system or device. A priming button 1137 can also be provided that would initiate a priming operation for the cooling wrap or cap. During the priming operation a priming wrap screen 1139 may also be provided that instructs the proper interactions with the reservoir.

In at least one example, the scalp cooling treatment screen 1107 or treatment session screen 1131 may also direct and/or provide a therapy setting confirmation screen 1141. The therapy setting confirmation screen 1141 can allow the user, patient, physician, or medical professional to review the pre-therapy cooling time and/or the post-therapy cooling time prior to initiating the treatment session. In at least one embodiment, an adjustment screen 1145 may be accessed if the review finds an error or a desired change. From the adjustment screen 1145 or the therapy setting confirmation screen 1141, the treatment session can be started in a treatment session screen 1143.

The treatment session screen 1143 can allow the user, patient, physician, or medical professional to know the remaining time, and/or the ability to pause the treatment session. A physician or medical professional may also provide a code or key to make changes to the treatment profile and/or settings. A first adjustment screen 1147 can allow a physician or medical professional to adjust the intensity and/or time for pre, during, and/or post session cooling. A second adjustment screen 1149 may also be provided once a change or adjustment has been made to allow a physician or medical professional to confirm the change or adjustment.

In at least one embodiment, any adjustment to the time for pre, during, and/or post session cooling would cause that portion of the session to start over again. A pause warning screen 1151 may be provided before any pauses, adjustments, and/or changes are made. The pause warning may let the user, patient, physician, or medical professional know that a timer reset can occur if the system is paused. In particular, if a pause occurs during the pre-cooling phase the timer can be reset due to the cooling of the cooling fluid to the desired temperature with the heat transfer assemblies. If the warning is accepted, the cooling system or device is paused and a pause screen 1153 may be provided to the user, patient, physician, or medical professional.

The pause screen 1153 may have a button such as a play button to allow a user to restart the cooling system or device. In at least one example, the pause screen 1153 may also indicates the time or countdown time that the system or device was paused at. After the system or device is unpaused, the system or device may begin the countdown or timing operation again from the time that it was paused at or starting over with a new time point. When the time had expired or countdown time has expired a pre-cooling complete screen 1155. In at least one example, the pre-cooling complete screen 1155 may stay displayed for a specified period before an adjustment screen 1157 is presented and/or displayed to a user. The adjustment screen 1157 may allow a user, patient, physician, or medical professional with the ability to make adjustments or changes to the cooling system or device before the infusion stage of a chemotherapy or other cancer treatment. In at least one example, the adjustment screen 1157 may allow for intensity or cooling point, and/or length of the time available for the cooling process. There may also be the ability for a user, patient, physician, or medical professional to pause or stop the cooling operation. For example, a user or patient may not be physically able to receive the chemotherapy or other cancer treatment, or may have become sick or need a moment of rest before continuing. Once the settings are approved or accepted, a ready to continue screen 1159 may also be provided to ensure that a user or patient is ready to move forward with both the chemotherapy or cancer treatment, and the cooling operation. In at least one embodiment, the ready to continue screen 1159 may have at least one button to allow for approval and/or a movement between the previous or next screen. If the user, patient, physician, or medical professional gives approval to begin the next stage of the treatment session than an active cooling screen 1161 would be provided. The active cooling screen 1161 may have a pause button and/or an indication of time available or time remaining.

Unlike the pre-cooling stage, a user, patient, physician, or medical professional may pause the active cooling stage, and then be provided an active cooling pause screen 1163. The active cooling pause screen 1163 may have a button to restart or resume the cooling operation, and/or indicate the time remaining or available for treatment. In at least one embodiment, a physician or medical professional may also make adjustments during the cooling operation via an adjustment screen 1165. In at least one example, the adjustment screen 1165 may have pause, stop, continue, and/or skip buttons in addition to various adjustment buttons such as, but not limited to a cooling intensity or temperature and/or time adjustments. If a skip is chosen or the time available or remaining has expired then a transition screen 1167 may be provided to a user, patient, physician, or medical professional. The transition screen 1167 allows for a pause or rest before beginning the next stage of the treatment. For example, the next stage of the treatment could be a post-therapy or treatment cool down period. After transition screen 1167, a post-therapy or infusion cooling screen 1169 may be provided. The post-therapy cooling screen 1160 may allow for the pausing and/or indication of the time available or remaining until the treatment sessions and/or cooling operation is completed. If a physician or medical professional desires to pause the treatment or cooling session, or make adjustments to the post-therapy operations of the cooling system or device, a post-therapy pause screen 1171 may be provided. The post-therapy pause screen 1171 may have at least one button to reactivate or resume the treatment or cooling session, and/or to access an adjustment screen 1173. The post-therapy pause screen 1171 may also have the ability to indicate the available or remain time for the treatment or cooling session. The adjustment screen 1173 can allow a physician or medical professional to modify the intensity, transition intensity or time, and/or the time remaining. In at least one embodiment, the adjustment screen 1173 can also have the ability to pause, stop, resume, and/or skip the post-therapy stage of the treatment or cooling session. At the conclusion of the treatment or cooling session, a treatment completed screen 1175 can be provided. The treatment completed screen 1175 can also provide a user or patient with instructions how to disconnect and/or notify a physician or medical professional that assistance is needed.

From the scalp cooling treatment screen 1107, if a physician or medical professional desires to make changes to the settings or system information they can be provided a set of screens for making such changes. One of these screens could be a session info screen 1177 that provides details about the specific session that is about to be authorized or activated. In at least one example, there may also be additional buttons provided on the screen that allow for transitioning to other screens. There may also be a therapy records screen 1179. In at least one embodiment, the therapy records screen 1179 contains information on a patient's therapy records with a cooling system or device, or other treatment or therapy. There may also be a treatment records screen 1181 that may contain information regarding the treatments provided by the cooling system or device. In at least one example, the treatment records screen 1181 may also provide patient information in a list form.

An alarm history screen 1183 can allow for a log or record of each alarm that has been and/or can be triggered by the cooling system or device. In at least one embodiment, the alarm history screen 1183 can allow for modification of the alarm levels (low, medium , and/or high) and/or notifications (visual, audible, push, text, and/or call) for each alarm level. A device usage screen 1185 may also provide information regarding the status, amount of use or session for an individual cooling system or device. In at least one embodiment, the device usage screen 1185 may also provide indications regarding session remaining for the service or maintenance of certain components or elements of the cooling system or device. A device system information screen 1187 may provide details such as serial number, operational levels, software version number, and/or update status. In at least one embodiment, the device system information screen 1187 can also allow for the update of the cooling system or device. A clock settings screen 1189 can allow visual configuration of the treatment screens. For example, the clock settings screen 1189 may allow for the time to be displayed in different styles such as, but not limited to, a count up timer, a countdown timer, or the clock time the session will be finished.

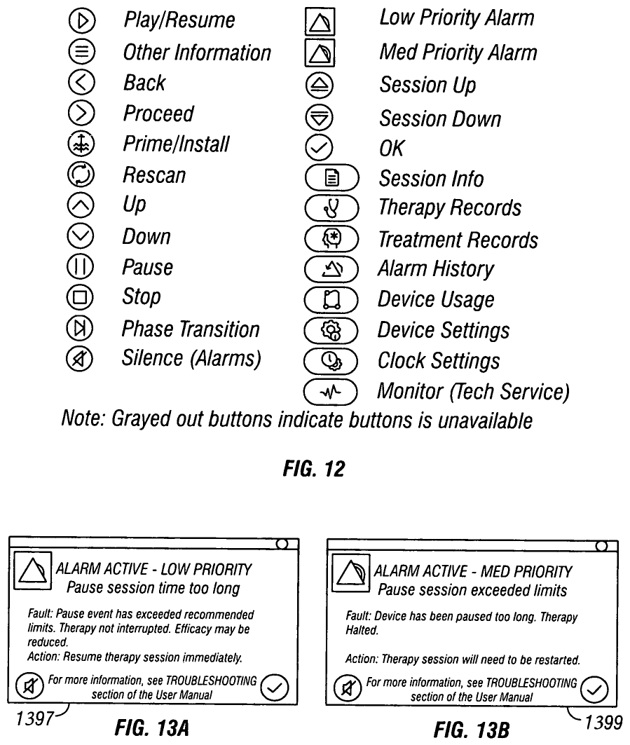

FIG. 12 is an illustration of the buttons and/or commands available with the cooling system or device. It would be understood that a reference to a button could include a visualization of button on a touch screen or a section of a touch screen that can be pressed or a similar like action such as, but not limited to, a capacitive reaction. Additionally, this illustrative list is not designed to represent a complete list of the available buttons and/or commands, additional commands and/or buttons that would represent these and other operations that may be discovered in the future would also be considered part of this disclosure.

FIGS. 13A and 13B are illustrative representations of the alarm screens that may be available and/or presented to a user, patient, physician, or medical professional if an alarm condition is triggered. FIG. 13A is an illustration of low priority alarm screen 1397. In at least one embodiment, the low priority alarm screen 1397 may include alerts for things such as low power, a pause in the session time exceeding a threshold, and/or a system update is available. FIG. 13B is an illustration of a medium priority alarm screen 1399. The medium priority alarm screen 1399 may be utilized to indicate when a therapy session has exceeded the maximum amount of time for a cooling operation, when the cooling fluid is below an initial level threshold, when a power failure has occurred, and/or when there is a cooling fluid blockage. Other additional alarms could include when a patient has disconnected the system or device, or when there has been an unauthorized attempt to access the cooling system or device.

While this disclosure has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

While various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, and by way of example, although the headings refer to a “Technical Field,” the claims should not be limited by the language chosen under this heading to describe the so-called field. Further, a description of a technology as background information is not to be construed as an admission that certain technology is prior art to any embodiment(s) in this disclosure. Neither is the “Brief Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

We claim:

1. An apparatus for cooling and transferring a fluid to a patient for cooling at least one portion of a patient's body comprising:
at least one heat transfer assembly for cooling the fluid;
a receiving point in fluid communication with the at least one heat transfer assembly;
a fluid transfer point in fluid communication with the at least one heat transfer assembly;
a hose for connecting the at least one fluid transfer point to an external device configured for attachment to the patient's body;
a temperature sensor coupled to the at least one fluid transfer point for monitoring a temperature of the fluid;
a controller connected to the temperature sensor for controlling the operation of the heat transfer assembly to maintain a selected temperature of the fluid.

2. The apparatus of claim 1, wherein the at least one sensor detects the temperature of a fluid traversing the at least one fluid transfer point.

3. The apparatus of claim 1, further comprising a computing device capable of receiving inputs from a user.

4. The apparatus of claim 1, further comprising a computing device coupled to a user interface that receives inputs from a user.

5. The apparatus of claim 1, further comprising a computing device coupled to a user interface that receives inputs from a user, the computing device controlling a flow of the fluid through the at least one fluid transfer point.

6. The apparatus of claim 1, further comprising a computing device coupled to a user interface that receives inputs from a user, the computing device connected to the controller.

7. A portable system for cooling and transferring a fluid to a patient for cooling at least one portion of a patient's body comprising:
an enclosure sized and configured to house at least one energy storage device;
at least one heat transfer assembly;
a fluid transfer point having a temperature sensor connected for monitoring a temperature of the fluid;
a fluid transfer hose having a first end coupled to the at least one fluid transfer point; and
a cooling wrap coupled to a second end of the fluid transfer hose.

8. The system of claim 7, wherein the cooling wrap receives the fluid from the fluid transfer hose.

9. The system of claim 7, wherein the cooling wrap returns a fluid to the fluid transfer hose after the fluid passes through a channel in the cooling wrap.

10. The system of claim 7, further comprising at least one computing device configured to communicate with the at least one heat transfer assembly, and the temperature sensor.

11. The system of claim 7, further comprising at least one computing device configured to communicate with the at least one heat transfer assembly and the temperature sensor and wherein the at least one computing device is coupled to a user interface.

12. The system of claim 7, further comprising at least one computing device configured to communicate with the at least one heat transfer assembly and the temperature sensor and wherein the at least one computing device is coupled to a user interface and the at least one computing device receives an input from the user interface.

13. The system of claim 7, further comprising at least one computing device configured to communicate with the at least one heat transfer assembly and the temperature sensor and wherein the at least one computing device is coupled to a user interface and the at least one computing device receives an input from the user interface for controlling the at least one heat transfer assembly.

14. The system of claim 7, wherein the temperature sensor is connected to the at least one heat transfer assembly allowing a temperature of a fluid passing through the heat transfer assembly to be changed based on detection of the temperature by the at least one sensor.

15. A method of cooling and transferring a fluid to medical device attached to a patient comprising:
filling a fluid receiving point with the fluid;
cooling the fluid with at least one heat transfer assembly;
transferring the fluid from the at least one heat transfer assembly to a cooling wrap through a fluid transfer hose;
receiving the fluid from the cooling wrap at the fluid transfer point; and
monitoring a fluid temperature at the fluid transfer point.

16. The method of claim 15, wherein the at least one heat transfer assembly removes heat from the fluid using at least one thermo-electric panel, allowing the fluid to be cooled.

17. The method of claim 15, wherein the monitoring steps are performed with at least one sensor.

18. The method of claim 15, wherein the monitoring step is performed by at least one computing device connected to a temperature sensor at the fluid transfer point.

19. The method of claim 15, wherein the monitoring step is performed by at least one computing device connected to a temperature sensor at the fluid transfer point and wherein the computing device controls the at least one heat transfer assembly based on a temperature signal received from the temperature sensor.

20. The method of claim 19 further comprising the step adjusting a temperature of the fluid with the at least one heat transfer assembly to maintain a consistent temperature of the fluid at the fluid transfer point.
